(19) United States
(12) Patent Application Publication
(10) Pub. No.: US 2005/0080055 A1
(43) Pub. Date: Apr. 14, 2005

(54) Method of treating breast cancer with androgen receptor antagonists

(76) Inventors: James T. Dalton, Upper Arlington, OH (US); Duane D. Miller, Germantown, TN (US); Mitchell S. Steiner, Germantown, TN (US); Karen A. Veverka, Cordova, TN (US)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/760,152

(22) Filed: Jan. 20, 2004

Related U.S. Application Data
(60) Provisional application No. 60/441,313, filed on Jan. 22, 2003.

(51) Int. Cl. A61K 31/66; A61K 31/32; A61K 31/165; A61K 31/277

(52) U.S. Cl. 514/114; 514/493; 514/522; 514/617; 514/618; 514/616

(57) ABSTRACT

This invention relates to the prevention and treatment of breast cancer in a subject, for example a female subject. Accordingly, this invention provides methods of a) treating a subject suffering from breast cancer; b) preventing, suppressing, inhibiting or reducing the incidence of breast cancer in a subject; c) delaying the progression of breast cancer in a subject suffering from breast cancer; d) preventing the recurrence of breast cancer in a subject; e) treating the recurrence of breast cancer in a subject suffering from breast cancer; and/or f) treating, preventing, suppressing or inhibiting metastasis in a subject suffering from breast cancer, by administering to the subject a therapeutically effective amount of an Androgen Receptor Antagonist and/or its analog, derivative, isomer, metabolite, pharmaceutically acceptable salt, pharmaceutical product, hydrate, N-oxide, crystal, polymorph, prodrug or any combination thereof, as described herein.

### CROSS REFERENCE TO RELATED APPLICATIONS

This application claims the priority of U.S. Provisional Application Ser. No. 60/441,313, filed Jan. 22, 2003, which is incorporated in its entirety by reference herein.

### FIELD OF INVENTION

The present invention relates to the prevention and treatment of breast cancer in a subject, for example a female subject. More particularly, this invention provides methods of a) treating a subject suffering from breast cancer; b) preventing, suppressing, inhibiting or reducing the incidence of breast cancer in a subject; c) delaying the progression of breast cancer in a subject suffering from breast cancer; d) preventing the recurrence of breast cancer in a subject; e) treating the recurrence of breast cancer in a subject suffering from breast cancer; and/or f) treating, preventing, suppressing or inhibiting metastasis in a subject suffering from breast cancer, by administering to the subject a therapeutically effective amount of an Androgen Receptor Antagonist and/or its analog, derivative, isomer, metabolite, pharmaceutically acceptable salt, pharmaceutical product, hydrate, N-oxide, crystal, polymorph, prodrug or any combination thereof, as described herein.

### BACKGROUND OF THE INVENTION

Breast cancer is a disease that kills over 45,000 women each year in the United States alone. Over 180,000 new cases of breast cancer are diagnosed annually, and it is estimated that one in eight women will develop breast cancer. These numbers indicate that breast cancer is one of the most dangerous diseases facing women today. Cancer research has been unable to determine the cause of breast cancer, and has not found a suitable method of therapy or prevention.

Currently, a woman diagnosed with breast cancer may be treated with surgery, hormone therapy, chemotherapy, and radiation. If the patient develops metastatic disease, radiation and high dose chemotherapy are required to ablate the cancer in remote areas such as the brain, bone, and liver.

The current therapies available for the treatment of breast cancer are toxic, dangerous, costly, and many are ineffective, especially in the treatment of metastatic disease. New innovative approaches are urgently needed at both the basic science and clinical levels to develop compounds which are useful for a) treating a subject suffering from breast cancer; b) preventing, suppressing, inhibiting or reducing the incidence of breast cancer in a subject; c) delaying the progression of breast cancer in a subject suffering from breast cancer; d) preventing the recurrence of breast cancer in a subject; e) treating the recurrence of breast cancer in a subject suffering from breast cancer; and/or f) treating, preventing, suppressing or inhibiting metastasis in a subject suffering from breast cancer.

### SUMMARY OF THE INVENTION

In one embodiment, this invention relates to the prevention and treatment of breast cancer in a subject, for example a female subject. Accordingly, this invention provides methods of a) treating a subject suffering from breast cancer; b) preventing, suppressing, inhibiting or reducing the incidence of breast cancer in a subject; c) delaying the progression of breast cancer in a subject suffering from breast cancer; d) preventing the recurrence of breast cancer in a subject; e) treating the recurrence of breast cancer in a subject suffering from breast cancer; and/or f) treating, preventing, suppressing or inhibiting metastasis in a subject suffering from breast cancer, by administering to the subject a therapeutically effective amount of an Androgen Receptor Antagonist and/or its analog, derivative, isomer, metabolite, pharmaceutically acceptable salt, pharmaceutical product, hydrate, N-oxide, crystal, polymorph, prodrug or any combination thereof, as described herein.

According to one embodiment of the present invention, a method is provided for treating a subject suffering from breast cancer, comprising the step of administering to the subject an Androgen Receptor Antagonist and/or its analog, derivative, isomer, metabolite, pharmaceutically acceptable salt, pharmaceutical product, hydrate, N-oxide, crystal, polymorph, prodrug or any combination thereof, in an amount effective to treat breast cancer in the subject. In one embodiment, the subject is a female subject. In another embodiment, the subject is a male subject.

According to another embodiment of the present invention, a method is provided for preventing breast cancer in a subject, comprising the step of administering to the subject an Androgen Receptor Antagonist and/or its analog, derivative, isomer, metabolite, pharmaceutically acceptable salt, pharmaceutical product, hydrate, N-oxide, crystal, polymorph, prodrug or any combination thereof, in an amount effective to treat prevent breast cancer in the subject. In one embodiment, the subject is a female subject. In another embodiment, the subject is a male subject.

According to another embodiment of the present invention, a method is provided for delaying the progression of breast cancer in a subject suffering from breast cancer, comprising the step of administering to the subject an Androgen Receptor Antagonist and/or its analog, derivative, isomer, metabolite, pharmaceutically acceptable salt, pharmaceutical product, hydrate, N-oxide, crystal, polymorph, prodrug or any combination thereof, in an amount effective to delay the progression of breast cancer in the subject. In one embodiment, the subject is a female subject. In another embodiment, the subject is a male subject.

According to another embodiment of the present invention, a method is provided for preventing the recurrence of breast cancer in a subject suffering from breast cancer, comprising the step of administering to the subject an Androgen Receptor Antagonist and/or its analog, derivative, isomer, metabolite, pharmaceutically acceptable salt, pharmaceutical product, hydrate, N-oxide, crystal, polymorph, prodrug or any combination thereof, in an amount effective to prevent the recurrence of breast cancer in the subject. In one embodiment, the subject is a female subject. In another embodiment, the subject is a male subject.

According to another embodiment of the present invention, a method is provided for treating the recurrence of breast cancer in a subject suffering from breast cancer, comprising the step of administering to the subject an Androgen Receptor Antagonist and/or its analog, derivative, isomer, metabolite, pharmaceutically acceptable salt, pharmaceutical product, hydrate, N-oxide, crystal, polymorph, prodrug or any combination thereof, in an amount effective to treat the recurrence of breast cancer in the subject. In one embodiment, the subject is a female subject. In another embodiment, the subject is a male subject.

According to another embodiment of the present invention, a method is provided for treating, preventing, suppressing or inhibiting metastasis in a subject suffering from breast cancer, comprising the step of administering to the subject an Androgen Receptor Antagonist and/or its analog, derivative, isomer, metabolite, pharmaceutically acceptable salt, pharmaceutical product, hydrate, N-oxide, crystal, polymorph, prodrug or any combination thereof, in an amount effective to treat, prevent, suppress or inhibit metastasis in the subject. In one embodiment, the subject is a female subject. In another embodiment, the subject is a male subject.

In one embodiment, the Androgen Receptor Antagonist that is effective at treating, preventing, delaying the progression of, preventing the recurrence of breast cancer and/or treating the recurrence of breast cancer, and/or treating, preventing, suppressing or inhibiting breast cancer metastasis is a compound represented by the structure of formula I, and/or its analog, derivative, isomer, metabolite, pharmaceutically acceptable salt, pharmaceutical product, hydrate, N-oxide, crystal, polymorph, prodrug or any combination thereof: 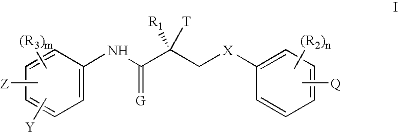
X is a bond, O, CH₂, NH, S, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 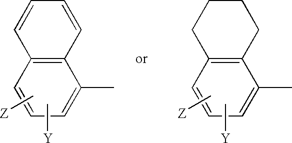
Z is NO₂, CN, COR, COOH, or CONHR;
Y is CF₃ F, Br, Cl, I, CN, or SnR₃;

Q is SCN, NCS, OCN, or NCO;

n is an integer of 1-4; and

m is an integer of 1-3.

In one embodiment, G in compound I is O, T is OH, R₁ is CH₃, X is O, Z is NO₂, Y is CF₃, and Q is NCS.

In another embodiment, the Androgen Receptor Antagonist that is effective at treating, preventing, delaying the progression of, preventing the recurrence of and/or treating the recurrence of breast cancer, and/or treating the recurrence of breast cancer, and/or treating, preventing, suppressing or inhibiting breast cancer metastasis is a compound represented by the structure of formula II, and/or its analog, derivative, isomer, metabolite, pharmaceutically acceptable salt, pharmaceutical product, hydrate, N-oxide, crystal, polymorph, prodrug or any combination thereof: 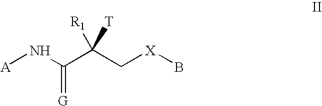
wherein X is a bond, O, CH₂, NH, S, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from: 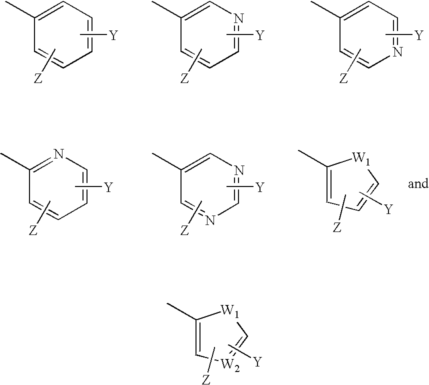
B is a ring selected from: 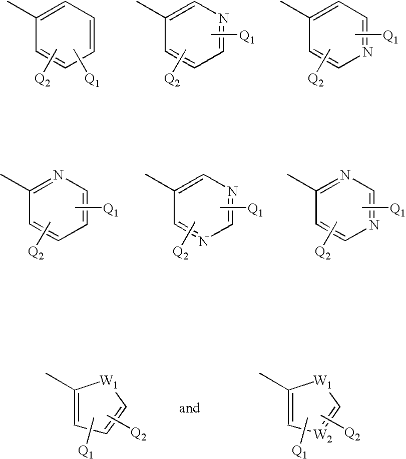
   wherein A and B cannot simultaneously be a benzene ring;
Z is NO₂, CN, COOH, COR, NHCOR or CONHR;
Y is CF₃, F, I, Br, Cl, CN CR₃ or SnR₃;
Q₁ is NCS, SCN, NCO or OCN;
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 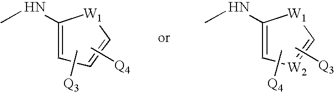
Q₃ and Q₄ are independently of each other a hydrogen, alkyl, halogen, CF₃, CN CR₃, SnR₃, NR₂, NHCOCH₃, NHCOCF₃, NHCOR, NHCON, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO.

In one embodiment, G in compound II is O, T is OH, R₁ is CH₃, X is O, Z is NO₂, Y is CF₃, and Q₁ is NCS.

In another embodiment, the Androgen Receptor Antagonist that is effective at treating, preventing, delaying the progression of, preventing the recurrence of and/or treating the recurrence of breast cancer, and/or treating the recurrence of breast cancer, and/or treating, preventing, suppressing or inhibiting breast cancer metastasis is a compound represented by the structure of formula III, and/or its analog, derivative, isomer, metabolite, pharmaceutically acceptable salt, pharmaceutical product, hydrate, N-oxide, crystal, polymorph, prodrug or any combination thereof: 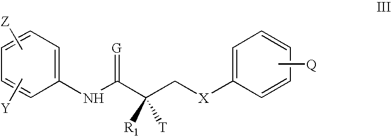
wherein X is a bond, O, CH₂, NH, S, Se, PR, NO or NR;
G is O or S;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is SCN, NCS, OCN, or NCO;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

In one embodiment, G in compound III is O, T is OH, R₁ is CH₃, X is O, Z is NO₂, Y is CF₃, and Q is NCS.

In another embodiment, the Androgen Receptor Antagonist that is effective at treating, preventing, delaying the progression of, preventing the recurrence of and/or treating the recurrence of breast cancer, and/or treating the recurrence of breast cancer, and/or treating, preventing, suppressing or inhibiting breast cancer metastasis is a compound represented by the structure of formula IV, and/or its analog, derivative, isomer, metabolite, pharmaceutically acceptable salt, pharmaceutical product, hydrate, N-oxide, crystal, polymorph, prodrug or any combination thereof: 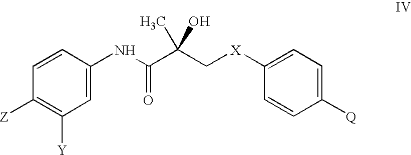
wherein X is a bond, O, CH₂, NH, S, Se, PR, NO or NR;
Z is NO₂, CN, COOH, COR, NHCOR or CONHR;
Y is CF₃, F, I, Br, Cl, CN, CR₃ or SnR₃;
Q is SCN, NCS, OCN, or NCO; and
R is alkyl, haloalkyl, dihaloalkyl, trihaloalkyl, CH₂F, CHF₂, CF₃, CF₂CF₃, aryl, phenyl, halogen, alkenyl or OH.

In one embodiment, X in compound IV is O, Z is NO₂, Y is CF₃, and Q is NCS.

In another embodiment, the Androgen Receptor Antagonist that is effective at treating, preventing, delaying the progression of, preventing the recurrence of and/or treating the recurrence of breast cancer, and/or treating the recurrence of breast cancer, and/or treating, preventing, suppressing or inhibiting breast cancer metastasis is a compound represented by the structure of formula V, and/or its analog, derivative, isomer, metabolite, pharmaceutically acceptable salt, pharmaceutical product, hydrate, N-oxide, crystal, polymorph, prodrug or any combination thereof:

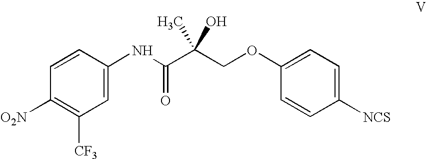

In one embodiment, the Androgen Receptor Antagonist of any of formulas I-V is an alkylating agent. In another embodiment, the Androgen Receptor Antagonist of any of formulas I-V binds irreversibly to an -androgen receptor. In another embodiment, the Androgen Receptor Antagonist of any of formulas I-V is an androgen receptor antagonist which binds irreversibly to an androgen receptor. In another embodiment, the Androgen Receptor Antagonist of any of formulas I-V is an alkylating agent.

The Androgen Receptor Antagonist compounds of the present invention, either alone or as a pharmaceutical composition, are useful for a) treating a subject suffering from breast cancer; b) preventing, suppressing, inhibiting or reducing the incidence of breast cancer in a subject; c) delaying the progression of breast cancer in a subject suffering from breast cancer; d) preventing the recurrence of breast cancer in a subject; e) treating the recurrence of breast cancer in a subject suffering from breast cancer; and/or treating, preventing, suppressing or inhibiting breast cancer metastasis.

The Androgen Receptor Antagonist compounds of the present invention offer a significant advance over steroidal androgen treatment since treatment of breast cancer with these compounds will not be accompanied by serious side effects, inconvenient modes of administration, or high costs and still have the advantages of oral bioavailability, lack of cross-reactivity with other steroid receptors, and long biological half-lives.

### BRIEF DESCRIPTION OF THE DRAWINGS

The present invention will be understood and appreciated more fully from the following detailed description taken in conjunction with the appended drawings:

FIG. 1: Growth curve demonstrating effect of Compound V on growth of LNCaP, CS LNCaP, PC-3, MCF-7, and CV-1 cell lines

FIG. 2: Growth inhibition of MCF-7 Breast Adenocarcinoma cells in presence of pure antiestrogen, ICI-182

### DETAILED DESCRIPTION OF THE INVENTION

In one embodiment, this invention relates to the prevention and treatment of breast cancer in a subject, for example a female subject. Accordingly, this invention provides methods of a) treating a subject suffering from breast cancer; b) preventing, suppressing, inhibiting or reducing the incidence of breast cancer in a subject; c) delaying the progression of breast cancer in a subject suffering from breast cancer; d) preventing the recurrence of breast cancer in a subject; e) treating the recurrence of breast cancer in a subject suffering from breast cancer; and/or f) treating, preventing, suppressing or inhibiting metastasis in a subject suffering from breast cancer, by administering to the subject a therapeutically effective amount of an Androgen Receptor Antagonist and/or its analog, derivative, isomer, metabolite, pharmaceutically acceptable salt, pharmaceutical product, hydrate, N-oxide, crystal, polymorph, prodrug or any combination thereof, as described herein.

According to one embodiment of the present invention, a method is provided for treating a subject suffering from breast cancer, comprising the step of administering to the subject an Androgen Receptor Antagonist and/or its analog, derivative, isomer, metabolite, pharmaceutically acceptable salt, pharmaceutical product, hydrate, N-oxide, crystal, polymorph, prodrug or any combination thereof, in an amount effective to treat breast cancer in the subject. In one embodiment, the subject is a female subject. In another embodiment, the subject is a male subject.

According to another embodiment of the present invention, a method is provided for preventing breast cancer in a subject, comprising the step of administering to the subject an Androgen Receptor Antagonist and/or its analog, derivative, isomer, metabolite, pharmaceutically acceptable salt, pharmaceutical product, hydrate, N-oxide, crystal, polymorph, prodrug or any combination thereof, in an amount effective to treat prevent breast cancer in the subject. In one embodiment, the subject is a female subject. In another embodiment, the subject is a male subject.

According to another embodiment of the present invention, a method is provided for delaying the progression of breast cancer in a subject suffering from breast cancer, comprising the step of administering to the subject an Androgen Receptor Antagonist and/or its analog, derivative, isomer, metabolite, pharmaceutically acceptable salt, pharmaceutical product, hydrate, N-oxide, crystal, polymorph, prodrug or any combination thereof, in an amount effective to delay the progression of breast cancer in the subject. In one embodiment, the subject is a female subject. In another embodiment, the subject is a male subject.

According to another embodiment of the present invention, a method is provided for preventing the recurrence of breast cancer in a subject suffering from breast cancer, comprising the step of administering to the subject an Androgen Receptor Antagonist and/or its analog, derivative, isomer, metabolite, pharmaceutically acceptable salt, pharmaceutical product, hydrate, N-oxide, crystal, polymorph, prodrug or any combination thereof, in an amount effective to prevent the recurrence of breast cancer in the subject. In one embodiment, the subject is a female subject. In another embodiment, the subject is a male subject.

According to another embodiment of the present invention, a method is provided for treating the recurrence of breast cancer in a subject suffering from breast cancer, comprising the step of administering to the subject an Androgen Receptor Antagonist and/or its analog, derivative, isomer, metabolite, pharmaceutically acceptable salt, pharmaceutical product, hydrate, N-oxide, crystal, polymorph, prodrug or any combination thereof, in an amount effective to treat the recurrence of breast cancer in the subject. In one embodiment, the subject is a female subject. In another embodiment, the subject is a male subject.

According to another embodiment of the present invention, a method is provided for treating, preventing, suppressing or inhibiting metastasis in a subject suffering from breast cancer, comprising the step of administering to the subject an Androgen Receptor Antagonist and/or its analog, derivative, isomer, metabolite, pharmaceutically acceptable salt, pharmaceutical product, hydrate, N-oxide, crystal, polymorph, prodrug or any combination thereof, in an amount effective to treat, prevent, suppress or inhibit metastasis in the subject. In one embodiment, the subject is a female subject. In another embodiment, the subject is a male subject.

In one embodiment, the Androgen Receptor Antagonist that is effective at treating, preventing, delaying the progression of, preventing the recurrence of and/or treating the recurrence of breast cancer, and/or treating, preventing, suppressing or inhibiting metastasis in a subject suffering from breast cancer, is a compound represented by the structure of formula I, and/or its analog, derivative, isomer, metabolite, pharmaceutically acceptable salt, pharmaceutical product, hydrate, N-oxide, crystal, polymorph, prodrug or any combination thereof: 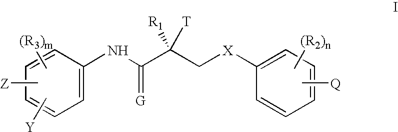
X is a bond, O, CH₂, NH, S, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 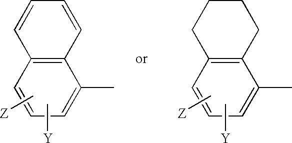
Z is NO₂, CN, COR, COOH, or CONHR;
Y is CF₃, F, Br, Cl, I, CN, or SnR₃;
Q is SCN, NCS, OCN, or NCO;
n is an integer of 1-4; and
m is an integer of 1-3.

In one embodiment, the methods of the present invention comprise administering an analog of the compound of formula I. In another embodiment, the methods of the present invention comprise administering a derivative of the compound of formula I. In another embodiment, the methods of the present invention comprise administering an isomer of the compound of formula I. In another embodiment, the methods of the present invention comprise administering a metabolite of the compound of formula I. In another embodiment, the methods of the present invention comprise administering a pharmaceutically acceptable salt of the compound of formula I. In another embodiment, the methods of the present invention comprise administering a pharmaceutical product of the compound of formula I. In another embodiment, the methods of the present invention comprise administering a hydrate of the compound of formula I. In another embodiment, the methods of the present invention comprise administering an N-oxide of the compound of formula I. In another embodiment, the methods of the present invention comprise administering a polymorph of the compound of formula I. In another embodiment, the methods of the present invention comprise administering a crystal of the compound of formula I. In another embodiment, the methods of the present invention comprise administering a prodrug of the compound of formula I. In another embodiment, the methods of the present invention comprise administering a combination of any of an analog, derivative, metabolite, isomer, pharmaceutically acceptable salt, pharmaceutical product, hydrate, N-oxide, polymorph, crystal or prodrug of the compound of formula I.

In one embodiment, G in compound I is O. In another embodiment, X in compound I is O. In another embodiment, T in compound I is OH. In another embodiment, R_{I} in compound I is CH₃. In another embodiment, Z in compound I is NO₂. In another embodiment, Z in compound I is CN. In another embodiment, Y in compound I is CF₃. In another embodiment, Q in compound I is NCS. In another embodiment, Q in compound I is in the para position. In another embodiment, Z in compound I is in the para position. In another embodiment, Y in compound I is in the meta position. In another embodiment, G in compound I is O, T is OH, R₁ is CH₃, X is O, Z is NO₂, Y is CF₃, and Q is NCS.

The substituents Z, Y and R₃ can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituents Q and R₂ can be in any position of the ring carrying these substituents (hereinafter “B ring”). In one embodiment, the substituent Q is in the para position of the B ring. In another embodiment, the substituent Q is in the para position of the B ring. In another embodiment, the substituent Q is NCS and is in the para position of the B ring.

As contemplated herein, when the integers m and n are greater than one, the substituents R₂ and R₃ are not limited to one particular substituent, and can be any combination of the substituents listed above.

In another embodiment, the Androgen Receptor Antagonist that is effective at treating, preventing, delaying the progression of, preventing the recurrence of and/or treating the recurrence of breast cancer, and/or treating, preventing, suppressing or inhibiting metastasis in a subject suffering from breast cancer, is a compound represented by the structure of formula II, and/or its analog, derivative, isomer, metabolite, pharmaceutically acceptable salt, pharmaceutical product, hydrate, N-oxide, crystal, polymorph, prodrug or any combination thereof: 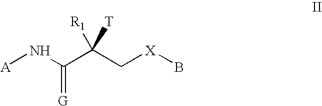
wherein X is a bond, O, CH₂, NH, S, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from: 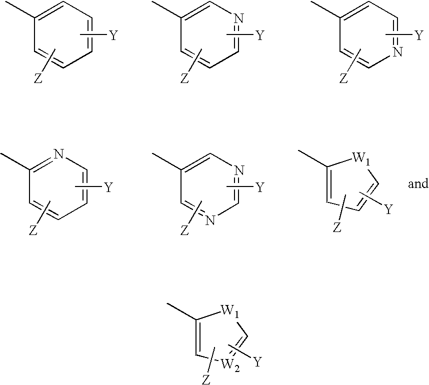
B is a ring selected from: 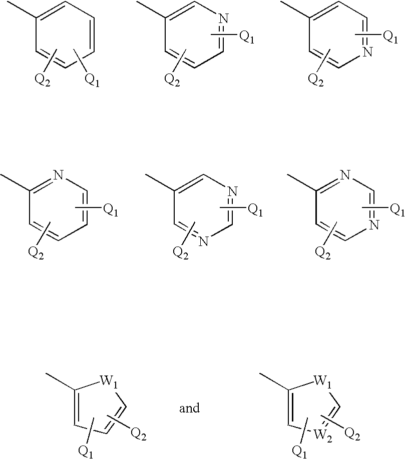
   wherein A and B cannot simultaneously be a benzene ring;
Z is NO₂, CN, COOH, COR, NHCOR or CONHR;
Y is CF₃, F, I, Br, Cl, CN CR₃ or SnR₃;
Q₁ is NCS, SCN, NCO or OCN;
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 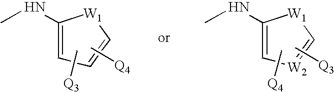
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO.

In one embodiment, the methods of the present invention comprise administering an analog of the compound of formula II. In another embodiment, the methods of the present invention comprise administering a derivative of the compound of formula II. In another embodiment, the methods of the present invention comprise administering an isomer of the compound of formula II. In another embodiment, the methods of the present invention comprise administering a metabolite of the compound of formula II. In another embodiment, the methods of the present invention comprise administering a pharmaceutically acceptable salt of the compound of formula II. In another embodiment, the methods of the present invention comprise administering a pharmaceutical product of the compound of formula II. In another embodiment, the methods of the present invention comprise administering a hydrate of the compound of formula II. In another embodiment, the methods of the present invention comprise administering an N-oxide of the compound of formula II. In another embodiment, the methods of the present invention comprise administering a polymorph of the compound of formula II. In another embodiment, the methods of the present invention comprise administering a crystal of the compound of formula II. In another embodiment, the methods of the present invention comprise administering a prodrug of the compound of formula II. In another embodiment, the methods of the present invention comprise administering a combination of any of an analog, derivative, metabolite, isomer, pharmaceutically acceptable salt, pharmaceutical product, hydrate, N-oxide, polymorph, crystal or prodrug of the compound of formula II.

In one embodiment, G in compound II is O. In another embodiment, X in compound II is O. In another embodiment, T in compound II is OH. In another embodiment, R₁ in compound II is CH₃. In another embodiment, Z in compound II is NO₂. In another embodiment, Z in compound II is CN. In another embodiment, Y in compound II is CF₃. In another embodiment, Q₁in compound II is NCS. In another embodiment, Q₁ in compound II is in the para position. In another embodiment, Z in compound II is in the para position. In another embodiment, Y in compound II is in the meta position. In another embodiment, G in compound II is O, T is OH, R₁ is CH₃, X is O, Z is NO₂, Y is CF₃, and Q₁ is NCS.

The substituents Z and Y can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituents Q₁ and Q₂ can be in any position of the ring carrying these substituents (hereinafter “B ring”). In one embodiment, the substituent Q₁ is in the para position of the B ring. In another embodiment, the substituent is Q₂ is H. In another embodiment, the substituent Q₁ is in the para position of the B ring and the substituent is Q₂ is H. In another embodiment, the substituent Q₁ is NCS and is in the para position of the B ring, and the substituent is Q₂ is H.

In another embodiment, the Androgen Receptor Antagonist that is effective at treating, preventing, delaying the progression of, preventing the recurrence of and/or treating the recurrence of breast cancer, and/or treating, preventing, suppressing or inhibiting metastasis in a subject suffering from breast cancer, is a compound represented by the structure of formula III, and/or its analog, derivative, isomer, metabolite, pharmaceutically acceptable salt, pharmaceutical product, hydrate, N-oxide, crystal, polymorph, prodrug or any combination thereof: 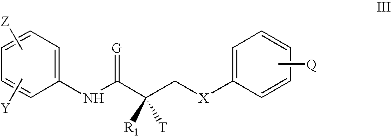
wherein X is a bond, O, CH₂, NH, S, Se, PR, NO or NR;
G is O or S;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is SCN, NCS, OCN, or NCO;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

In one embodiment, the methods of the present invention comprise administering an analog of the compound of formula III. In another embodiment, the methods of the present invention comprise administering a derivative of the compound of formula III. In another embodiment, the methods of the present invention comprise administering an isomer of the compound of formula III. In another embodiment, the methods of the present invention comprise administering a metabolite of the compound of formula III. In another embodiment, the methods of the present invention comprise administering a pharmaceutically acceptable salt of the compound of formula III. In another embodiment, the methods of the present invention comprise administering a pharmaceutical product of the compound of formula III. In another embodiment, the methods of the present invention comprise administering a hydrate of the compound of formula III. In another embodiment, the methods of the present invention comprise administering an N-oxide of the compound of formula III. In another embodiment, the methods of the present invention comprise administering a polymorph of the compound of formula III. In another embodiment, the methods of the present invention comprise administering a crystal of the compound of formula III. In another embodiment, the methods of the present invention comprise administering a prodrug of the compound of formula III. In another embodiment, the methods of the present invention comprise administering a combination of any of an analog, derivative, metabolite, isomer, pharmaceutically acceptable salt, pharmaceutical product, hydrate, N-oxide, polymorph, crystal or prodrug of the compound of formula III.

In one embodiment, G in compound III is O. In another embodiment, X in compound III is O. In another embodiment, T in compound III is OH. In another embodiment, R₁ in compound III is CH₃. In another embodiment, Z in compound III is NO₂. In another embodiment, Z in compound III is CN. In another embodiment, Y in compound III is CF₃. In another embodiment, Q in compound III is NCS. In another embodiment, Q in compound III is in the para position. In another embodiment, Z in compound III is in the para position. In another embodiment, Y in compound III is in the meta position. In another embodiment, G in compound III is O, T is OH, R₁ is CH₃, X is O, Z is NO₂, Y is CF₃, and Q is NCS.

The substituents Z and Y can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituent Q can be in any position of the ring carrying this substituent (hereinafter “B ring”). In one embodiment, the substituent Q is in the para position of the B ring. In another embodiment, the substituent Q is NCS and is in the para position of the B ring.

In another embodiment, the Androgen Receptor Antagonist that is effective at treating, preventing, delaying the progression of, preventing the recurrence of and/or treating the recurrence of breast cancer, and/or treating, preventing, suppressing or inhibiting metastasis in a subject suffering from breast cancer, is a compound represented by the structure of formula IV, and/or its analog, derivative, isomer, metabolite, pharmaceutically acceptable salt, pharmaceutical product, hydrate, N-oxide, crystal, polymorph, prodrug or any combination thereof: 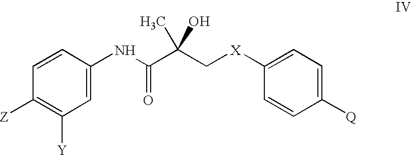
wherein X is a bond, O, CH₂, NH, S, Se, PR, NO or NR;
Z is NO₂, CN, COOH, COR, NHCOR or CONHR;
Y is CF₃, F, I, Br, Cl, CN, CR₃ or SnR₃;
Q is SCN, NCS, OCN, or NCO; and
R is alkyl, haloalkyl, dihaloalkyl, trihaloalkyl, CH₂F, CHF₂, CF₃, CF₂CF₃, aryl, phenyl, halogen, alkenyl or OH.

In one embodiment, the methods of the present invention comprise administering an analog of the compound of formula IV. In another embodiment, the methods of the present invention comprise administering a derivative of the compound of formula IV. In another embodiment, the methods of the present invention comprise administering an isomer of the compound of formula IV. In another embodiment, the methods of the present invention comprise administering a metabolite of the compound of formula IV. In another embodiment, the methods of the present invention comprise administering a pharmaceutically acceptable salt of the compound of formula IV. In another embodiment, the methods of the present invention comprise administering a pharmaceutical product of the compound of formula IV. In another embodiment, the methods of the present invention comprise administering a hydrate of the compound of formula IV. In another embodiment, the methods of the present invention comprise administering an N-oxide of the compound of formula IV. In another embodiment, the methods of the present invention comprise administering a polymorph of the compound of formula IV. In another embodiment, the methods of the present invention comprise administering a crystal of the compound of formula IV. In another embodiment, the methods of the present invention comprise administering a prodrug of the compound of formula IV. In another embodiment, the methods of the present invention comprise administering a combination of any of an analog, derivative, metabolite, isomer, pharmaceutically acceptable salt, pharmaceutical product, hydrate, N-oxide, polymorph, crystal or prodrug of the compound of formula IV.

In one embodiment, X in compound IV is O. In another embodiment, Z in compound IV is NO₂. In another embodiment, Z in compound IV is CN. In another embodiment, Y in compound IV is CF₃. In another embodiment, Q in compound IV is NCS. In another embodiment, X in compound IV is O, Z is NO₂, Y is CF₃, and Q is NCS.

In another embodiment, the Androgen Receptor Antagonist that is effective at treating, preventing, delaying the progression of, preventing the recurrence of and/or treating the recurrence of breast cancer, and/or treating, preventing, suppressing or inhibiting metastasis in a subject suffering from breast cancer, is a compound represented by the structure of formula V, and/or its analog, derivative, isomer, metabolite, pharmaceutically acceptable salt, pharmaceutical product, hydrate, N-oxide, crystal, polymorph, prodrug or any combination thereof:

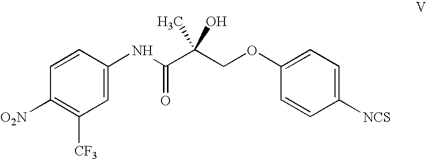

In one embodiment, the methods of the present invention comprise administering an analog of the compound of formula V. In another embodiment, the methods of the present invention comprise administering a derivative of the compound of formula V. In another embodiment, the methods of the present invention comprise administering an isomer of the compound of formula V. In another embodiment, the methods of the present invention comprise administering a metabolite of the compound of formula V. In another embodiment, the methods of the present invention comprise administering a pharmaceutically acceptable salt of the compound of formula V. In another embodiment, the methods of the present invention comprise administering a pharmaceutical product of the compound of formula V. In another embodiment, the methods of the present invention comprise administering a hydrate of the compound of formula V. In another embodiment, the methods of the present invention comprise administering an N-oxide of the compound of formula V. In another embodiment, the methods of the present invention comprise administering a polymorph of the compound of formula V. In another embodiment, the methods of the present invention comprise administering a crystal of the compound of formula V. In another embodiment, the methods of the present invention comprise administering a prodrug of the compound of formula V. In another embodiment, the methods of the present invention comprise administering a combination of any of an analog, derivative, metabolite, isomer, pharmaceutically acceptable salt, pharmaceutical product, hydrate, N-oxide, polymorph, crystal or prodrug of the compound of formula V.

As contemplated herein, other specific embodiments of compounds included within the scope of the present invention, and which are useful in the treatment/prevention of breast cancer, are compounds VI and VII. It is understood that included within the scope of the present invention are analogs, derivatives, metabolites, isomers, pharmaceutically acceptable salts, pharmaceutical products, hydrates, N-oxides, polymorphs, crystals, prodrugs or combinations thereof of these compounds:

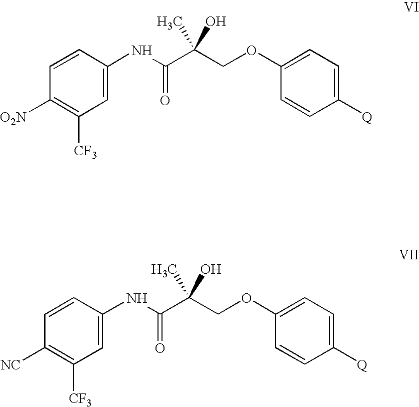

wherein Q is NCS, SCN, NCO or OCN.

In one embodiment, the methods of the present invention comprise administering an analog of the compound of formula VI. In another embodiment, the methods of the present invention comprise administering a derivative of the compound of formula VI. In another embodiment, the methods of the present invention comprise administering an isomer of the compound of formula VI. In another embodiment, the methods of the present invention comprise administering a metabolite of the compound of formula VI. In another embodiment, the methods of the present invention comprise administering a pharmaceutically acceptable salt of the compound of formula VI. In another embodiment, the methods of the present invention comprise administering a pharmaceutical product of the compound of formula VI. In another embodiment, the methods of the present invention comprise administering a hydrate of the compound of formula VI. In another embodiment, the methods of the present invention comprise administering an N-oxide of the compound of formula VI. In another embodiment, the methods of the present invention comprise administering a polymorph of the compound of formula VI. In another embodiment, the methods of the present invention comprise administering a crystal of the compound of formula VI. In another embodiment, the methods of the present invention comprise administering a prodrug of the compound of formula VI. In another embodiment, the methods of the present invention comprise administering a combination of any of an analog, derivative, metabolite, isomer, pharmaceutically acceptable salt, pharmaceutical product, hydrate, N-oxide, polymorph, crystal or prodrug of the compound of formula VI.

In one embodiment, the methods of the present invention comprise administering an analog of the compound of formula VII. In another embodiment, the methods of the present invention comprise administering a derivative of the compound of formula VII. In another embodiment, the methods of the present invention comprise administering an isomer of the compound of formula VII. In another embodiment, the methods of the present invention comprise administering a metabolite of the compound of formula VII. In another embodiment, the methods of the present invention comprise administering a pharmaceutically acceptable salt of the compound of formula VII. In another embodiment, the methods of the present invention comprise administering a pharmaceutical product of the compound of formula VII. In another embodiment, the methods of the present invention comprise administering a hydrate of the compound of formula VII. In another embodiment, the methods of the present invention comprise administering an N-oxide of the compound of formula VII. In another embodiment, the methods of the present invention comprise administering a polymorph of the compound of formula VII. In another embodiment, the methods of the present invention comprise administering a crystal of the compound of formula VII. In another embodiment, the methods of the present invention comprise administering a prodrug of the compound of formula VII. In another embodiment, the methods of the present invention comprise administering a combination of any of an analog, derivative, metabolite, isomer, pharmaceutically acceptable salt, pharmaceutical product, hydrate, N-oxide, polymorph, crystal or prodrug of the compound of formula VII.

The substituent R is defined herein as an alkyl, haloalkyl, dihaloalkyl, trihaloalkyl, CH₂F, CHF₂, CF₃, CF₂CF₃; aryl, phenyl, halogen, alkenyl, or hydroxyl (OH).

An “alkyl” group refers to a saturated aliphatic hydrocarbon, including straight-chain, branched-chain and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. The alkyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl.

A “haloalkyl” group refers to an alkyl group as defined above, which is substituted by one or more halogen atoms, e.g. by F, Cl, Br or I.

An “aryl” group refers to an aromatic group having at least one carbocyclic aromatic group or heterocyclic aromatic group, which may be unsubstituted or substituted by one or more groups selected from halogen, haloalkyl, hydroxy, alkoxy carbonyl, amido, alkylamido, dialkylamido, nitro, amino, alkylamino, dialkylamino, carboxy or thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like.

A “hydroxyl” group refers to an OH group. An “alkenyl” group refers to a group having at least one carbon to carbon double bond. A halo group refers to F, Cl, Br or I.

An “arylalkyl” group refers to an alkyl bound to an aryl, wherein alkyl and aryl are as defined above. An example of an aralkyl group is a benzyl group.

As contemplated herein, the present invention relates to the use of an Androgen Receptor Antagonist compound and/or its analog, derivative, isomer, metabolite, pharmaceutically acceptable salt, pharmaceutical product, hydrate, N-oxide, prodrug, polymorph, crystal or combinations thereof. In one embodiment, the invention relates to the use of an analog of the Androgen Receptor Antagonist. In another embodiment, the invention relates to the use of a derivative of the Androgen Receptor Antagonist. In another embodiment, the invention relates to the use of an isomer of the Androgen Receptor Antagonist. In another embodiment, the invention relates to the use of a metabolite of the Androgen Receptor Antagonist. In another embodiment, the invention relates to the use of a pharmaceutically acceptable salt of the Androgen Receptor Antagonist. In another embodiment, the invention relates to the use of a pharmaceutical product of the Androgen Receptor Antagonist. In another embodiment, the invention relates to the use of a hydrate of the Androgen Receptor Antagonist. In another embodiment, the invention relates to the use of an N-oxide of the Androgen Receptor Antagonist. In another embodiment, the invention relates to the use of a polymorph of the Androgen Receptor Antagonist. In another embodiment, the invention relates to the use of a crystal of the Androgen Receptor Antagonist. In another embodiment, the invention relates to the use of a prodrug of the Androgen Receptor Antagonist. In another embodiment, the invention relates to the use of any of a combination of an analog, derivative, isomer, metabolite, pharmaceutically acceptable salt, pharmaceutical product, hydrate, N-oxide, polymorph, crystal or prodrug of the Androgen Receptor Antagonists of the present invention.

As defined herein, the term “isomer” includes, but is not limited to, optical isomers and analogs, structural isomers and analogs, conformational isomers and analogs, and the like.

In one embodiment, this invention encompasses the use of various optical isomers of the Androgen Receptor Antagonist. It will be appreciated by those skilled in the art that the Androgen Receptor Antagonist of the present invention contain at least one chiral center. Accordingly, the Androgen Receptor Antagonists used in the methods of the present invention may exist in, and be isolated in, optically-active or racemic forms. Some compounds may also exhibit polymorphism. It is to be understood that the present invention encompasses any racemic, optically-active, polymorphic, or stereroisomeric form, or any combination thereof, which form possesses properties useful in the treatment of androgen-related conditions described herein. In one embodiment, the Androgen Receptor Antagonists are the pure (R)-isomers. In another embodiment, the Androgen Receptor Antagonists are the pure (S)-isomers. In another embodiment, the Androgen Receptor Antagonists are a mixture of the (R) and the (S) isomers. In another embodiment, the Androgen Receptor Antagonists are a racemic mixture comprising an equal amount of the (R) and the (S) isomers. It is well known in the art how to prepare optically-active forms (for example, by resolution of the racemic form by recrystallization techniques, by synthesis from optically-active starting materials, by chiral synthesis, or by chromatographic separation using a chiral stationary phase).

The invention includes pharmaceutically acceptable salts of amino-substituted compounds with organic and inorganic acids, for example, citric acid and hydrochloric acid. The invention also includes N-oxides of the amino substituents of the compounds described herein. Pharmaceutically acceptable salts can also be prepared from the phenolic compounds by treatment with inorganic bases, for example, sodium hydroxide. Also, esters of the phenolic compounds can be made with aliphatic and aromatic carboxylic acids, for example, acetic acid and benzoic acid esters.

This invention further includes derivatives of the Androgen Receptor Antagonists. The term “derivatives” includes but is not limited to ether derivatives, acid derivatives, amide derivatives, ester derivatives and the like. In addition, this invention further includes hydrates of the Androgen Receptor Antagonists. The term “hydrate” includes but is not limited to hemihydrate, monohydrate, dihydrate, trihydrate and the like.

This invention further includes metabolites of the Androgen Receptor Antagonists. The term “metabolite” means any substance produced from another substance by metabolism or a metabolic process.

This invention further includes pharmaceutical products of the Androgen Receptor Antagonists. The term “pharmaceutical product” means a composition suitable for pharmaceutical use (pharmaceutical composition), as defined herein.

This invention further includes prodrugs of the Androgen Receptor Antagonists. The term “prodrug” means a substance which can be converted in-vivo into a biologically active agent by such reactions as hydrolysis, esterification, desterification, activation, salt formation and the like.

This invention further includes crystals of the Androgen Receptor Antagonists. Furthermore, this invention provides polymorphs of the Androgen Receptor Antagonists. The term “crystal” means a substance in a crystalline state. The term “polymorph” refers to a particular crystalline state of a substance, having particular physical properties such as X-ray diffraction, IR spectra, melting point, and the like.

Biological Activity of Androgen Receptor Antagonists

The Androgen Receptor Antagonists provided herein are a new class of compounds which bind irreversibly to the Androgen Receptor. Several of the compounds have an unexpected antiandrogenic activity of a nonsteroidal ligand for the androgen receptor. Furthermore several of the compounds of the present invention are alkylating agents. In addition, in one embodiment, the Androgen Receptor Antagonists of the present invention are Selective Androgen Receptor Modulator (SARM) compounds.

As contemplated herein, the appropriately substituted Androgen Receptor Antagonists of the present invention are useful for a) treating a subject suffering from breast cancer; b) preventing, suppressing, inhibiting or reducing the incidence of breast cancer in a subject; c) delaying the progression of breast cancer in a subject suffering from breast cancer; d) preventing the recurrence of breast cancer in a subject; e) treating the recurrence of breast cancer in a subject suffering from breast cancer; and/or f) treating, preventing, suppressing or inhibiting metastasis in a subject suffering from breast cancer.

As used herein, receptors for extracellular signaling molecules are collectively referred to as “cell signaling receptors”. Many cell signaling receptors are transmembrane proteins on a cell surface; when they bind an extracellular signaling molecule (i.e., a ligand), they become activated so as to generate a cascade of intracellular signals that alter the behavior of the cell. In contrast, in some cases, the receptors are inside the cell and the signaling ligand has to enter the cell to activate them; these signaling molecules therefore must be sufficiently small and hydrophobic to diffuse across the plasma membrane of the cell.

Steroid hormones are one example of small hydrophobic molecules that diffuse directly across the plasma membrane of target cells and bind to intracellular cell signaling receptors. These receptors are structurally related and constitute the intracellular receptor superfamily (or steroid-hormone receptor superfamily). Steroid hormone receptors include but are not limited to progesterone receptors, estrogen receptors, androgen receptors, glueocorticoid receptors, and mineralocorticoid receptors. In one embodiment, the present invention is directed to androgen receptors.

In addition to ligand binding to the receptors, the receptors can be blocked to prevent ligand binding. When a substance binds to a receptor, the three-dimensional structure of the substance fits into a space created by the three-dimensional structure of the receptor in a ball and socket configuration. The better the ball fits into the socket, the more tightly it is held. This phenomenon is called affinity. If the affinity of a substance is greater than the original hormone, it will compete with the hormone and bind the binding site more frequently. Once bound, signals may be sent through the receptor into the cells, causing the cell to respond in some fashion. This is called activation. On activation, the activated receptor then directly regulates the transcription of specific genes. But the substance and the receptor may have certain attributes, other than affinity, in order to activate the cell. Chemical bonds between atoms of the substance and the atoms of the receptors may form. In some cases, this leads to a change in the configuration of the receptor, which is enough to begin the activation process (called signal transduction).

A receptor antagonist is a substance which binds receptors and inactivates them. An Androgen Receptor Antagonist is a molecule that blocks signaling activity of the Androgen Receptor. Thus, in one embodiment, the Androgen Receptor Antagonists of the present invention are useful in binding to and inactivating steroidal hormone receptors. In one embodiment, the antagonist compound of the present invention is an antagonist which binds the androgen receptor. In another embodiment, the compound has high affinity for the androgen receptor.

Assays to determine whether the compounds of the present invention are AR agonists or antagonists are well known to a person skilled in the art. For example, AR agonistic activity can be determined by monitoring the ability of the Androgen Receptor Antagonists to maintain and/or stimulate the growth of AR containing tissue such as prostate and seminal vesicles, as measured by weight. AR antagonistic activity can be determined by monitoring the ability of the Androgen Receptor Antagonists to inhibit the growth of AR containing tissue.

An androgen receptor is an androgen receptor of any species, for example a mammal. In one embodiment, the androgen receptor is an androgen receptor of a human. Thus, in another embodiment, the Androgen Receptor Antagonists bind irreversibly to an androgen receptor of a human. In another embodiment, the Androgen Receptor Antagonists bind irreversibly to an androgen receptor of a mammal.

As contemplated herein, the term “androgen receptor antagonist” refers to, in one embodiment, a ligand-receptor binding interaction which prevents activation of the receptor by agonist by interacting in such a manner that it is bound tighter than the native ligand. In another embodiment, “androgen receptor antagonist” refers to another embodiment to a ligand-receptor binding interaction which prevents activation of the receptor by agonist by interacting in such a manner that cannot be reversed, i.e. the ligand, once bound, cannot detach from the receptor. Without being limited to any theory, an example of an irreversible binding interaction is a covalent bond formed between the ligand and the receptor. Such a covalent bond is formed when the ligand is an alkylating agent, i.e. contains a functional group that allows alkylation of the target receptor. Thus, in one embodiment, the compounds of the present invention may contain a functional group (e.g. affinity label) that allows alkylation of the androgen receptor (i.e. covalent bond formation). Thus, in this case, the compounds are alkylating agents which bind irreversibly to the receptor and, accordingly, cannot be displaced by a steroid, such as the endogenous ligands DHT and testosterone. An “alkylating agent” is defined herein as an agent which alkylates (forms a covalent bond) with a cellular component, such as DNA, RNA or enzyme. It is a highly reactive chemical that introduces alkyl radicals into biologically active molecules and thereby prevents their proper functioning. The alkylating moiety is an electrophilic group that interacts with nucleophilic moieties in cellular components. For example, in one embodiment, an alkylating group is an isocyanate moiety, an electrophilic group which forms covalent bonds with nucleophilic groups (N, O, S etc.) in cellular components. In another embodiment, an alkylating group is an isothiocyanate moiety, another electrophilic group which forms covalent bonds with nucleophilic groups (N, O, S etc.) in cellular components. In another embodiment, an alkylating group is a haloalkyl (CH₂X wherein X is halogen), an electrophilic group which forms covalent bonds with nucleophilic groups in cellular components. In another embodiment, an alkylating group is a haloalkyl-amido (NHCOCH₂X wherein X is halogen), an electrophilic group which forms covalent bonds with nucleophilic groups in cellular components.

The Androgen Receptor Antagonists are compounds which bind irreversibly to the androgen receptor. In one embodiment, the Androgen Receptor Antagonists of the present invention are androgen receptor antagonists which bind irreversibly to the androgen receptor of a mammal, for e.g. a human. In one embodiment, the compounds are alkylating agents.

The mechanism of action by which the Androgen Receptor Antagonists exert their biological effect does not limit the broad scope of the invention. Not wishing to be bound by theory, the mechanism of action by which the Androgen Receptor Antagonists exert their effects may be one or more of the following non-limiting list: a) irreversibly or covalently modifying active site of an enzyme to prevent action of endogenous or exogenous hormones b) irreversibly or covalently modifying an enzyme to prevent it from interacting with transcription elements in DNA; c) irreversibly or covalently modifying DNA with ligand-bound receptor to prevent it from interacting with nuclear hormone; receptors, transcription factors; d) nonspecifically alkylating DNA; e) blocking nonspecific cross-talk by peptide growth factors and other hormones; and/or f) binding to receptor and preventing nuclear translocation of the receptor.

As defined herein, “contacting” means that the Androgen Receptor Antagonists of the present invention is introduced into a sample containing the enzyme in a test tube, flask, tissue culture, chip, array, plate, microplate, capillary, or the like, and incubated at a temperature and time sufficient to permit binding of the Androgen Receptor Antagonists to the enzyme. Methods for contacting the samples with the Androgen Receptor Antagonists or other specific binding components are known to those skilled in the art and may be selected depending on the type of assay protocol to be run. Incubation methods are also standard and are known to those skilled in the art.

In another embodiment, the term “contacting” means that the Androgen Receptor Antagonists of the present invention are introduced into a subject receiving treatment, and the Androgen Receptor Antagonist is allowed to come in contact with the androgen receptor in vivo.

As used herein, the term “treating” includes preventative as well as disorder remitative treatment. As used herein, the terms “reducing”, “suppressing” and “inhibiting” have their commonly understood meaning of lessening or decreasing. As used herein, the term “progression” means increasing in scope or severity, advancing, growing or becoming worse. As used herein, the term “recurrence” means the return of a disease after a remission. As used herein, the term “delaying” means stopping, hindering, slowing down, postponing, holding up or setting back. As used herein, the term “metastasis” refers to the transfer of a disease from one organ or part thereof to another not directly connected with it. Metastasis can occur for example as a result of transfer of malignant cells from one organ (for example breast) to other organs.

As used herein, the term “administering” refers to bringing a subject in contact with a compound of the present invention. As used herein, administration can be accomplished in vitro, i.e. in a test tube, or in vivo, i.e. in cells or tissues of living organisms, for example humans. In one embodiment, the present invention encompasses administering the compounds of the present invention to a subject.

In one embodiment, the methods of the present invention comprise administering an Androgen Receptor Antagonist as the sole active ingredient. However, also encompassed within the scope of the present invention are methods for hormone therapy, for treating breast cancer, for delaying the progression of breast cancer, and for preventing and treating the recurrence of breast cancer and/or breast cancer metastasis, which comprise administering the Androgen Receptor Antagonists in combination with one or more therapeutic agents. These agents include, but are not limited to: LHRH analogs, reversible antiandrogens, antiestrogens, anticancer drugs, 5-alpha reductase inhibitors, aromatase inhibitors, progestins, agents acting through other nuclear hormone receptors, selective estrogen receptor modulators (SERM), progesterone, estrogen, PDE5 inhibitors, apomorphine, bisphosphonate, growth factor inhibitors (such as those that inhibit VEGF, IGF and the like), a selective androgen receptor modulator (SARM) and one or more additional Androgen Receptor Antagonist.

Thus, in one embodiment, the methods of the present invention comprise administering the Androgen Receptor Antagonist, in combination with an LHRH analog. In another embodiment, the methods of the present invention comprise administering an Androgen Receptor Antagonist, in combination with a reversible antiandrogen. In another embodiment, the methods of the present invention comprise administering an Androgen Receptor Antagonist, in combination with an antiestrogen. In another embodiment, the methods of the present invention comprise administering an Androgen Receptor Antagonist, in combination with an anticancer drug. In another embodiment, the methods of the present invention comprise administering an Androgen Receptor Antagonist, in combination with a 5-alpha reductase inhibitor. In another embodiment, the methods of the present invention comprise administering an Androgen Receptor Antagonist, in combination with an aromatase inhibitor. In another embodiment, the methods of the present invention comprise administering an Androgen Receptor Antagonist, in combination with a progestin. In another embodiment, the methods of the present invention comprise administering an Androgen Receptor Antagonist, in combination with an agent acting through other nuclear hormone receptors. In another embodiment, the methods of the present invention comprise administering an Androgen Receptor Antagonist, in combination with a selective estrogen receptor modulators (SERM). In another embodiment, the methods of the present invention comprise administering an Androgen Receptor Antagonist, in combination with a progesterone. In another embodiment, the methods of the present invention comprise administering an Androgen Receptor Antagonist, in combination with an estrogen. In another embodiment, the methods of the present invention comprise administering an Androgen Receptor Antagonist, in combination with a PDE5 inhibitor. In another embodiment, the methods of the present invention comprise administering an Androgen Receptor Antagonist, in combination with apomorphine. In another embodiment, the methods of the present invention comprise administering an Androgen Receptor Antagonist, in combination with a bisphosphonate. In another embodiment, the methods of the present invention comprise administering an Androgen Receptor Antagonist, in combination with a growth factor inhibitor. In another embodiment, the methods of the present invention comprise administering an Androgen Receptor Antagonist, in combination with a selective androgen receptor modulator (SARM). In another embodiment, the methods of the present invention comprise administering an Androgen Receptor Antagonist, in combination with one or more additional Androgen Receptor Antagonist.

In one embodiment, the methods of the present invention comprise administering a pharmaceutical composition (or pharmaceutical preparation, used herein interchangeably) comprising the Androgen Receptor Antagonist of the present invention and/or its analog, derivative, isomer, metabolite, pharmaceutical product, hydrate, N-oxide, polymorph, crystal, prodrug or any combination thereof; and a suitable carrier or diluent.

Pharmaceutical Compositions:

As used herein, “pharmaceutical composition” means therapeutically effective amounts of the Androgen Receptor Antagonist together with suitable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or Lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance. Controlled or sustained release compositions include formulation in lipophilic depots (e.g., fatty acids, waxes, oils).

Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitonealy, intraventricularly, intravaginally, intracranially and intratumorally.

Further, as used herein “pharmaceutically acceptable carriers” are well known to those skilled in the art and include, but are not limited to, 0.01-0.1M and preferably 0.05M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

Other embodiments of the compositions of the invention incorporate particulate forms, protective coatings, protease inhibitors or permeation enhancers for various routes of administration, including parenteral, pulmonary, nasal and oral.

Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

The pharmaceutical preparation can comprise the Androgen Receptor Antagonist alone, or can further include a pharmaceutically acceptable carrier, and can be in solid or liquid form such as tablets, powders, capsules, pellets, solutions, suspensions, elixirs, emulsions, gels, creams, or suppositories, including rectal and urethral suppositories. Pharmaceutically acceptable carriers include gums, starches, sugars, cellulosic materials, and mixtures thereof. The pharmaceutical preparation containing the Androgen Receptor Antagonist can be administered to a subject by, for example, subcutaneous implantation of a pellet; in a further embodiment, the pellet provides for controlled release of Androgen Receptor Antagonist over a period of time. The preparation can also be administered by intravenous, intraarterial, or intramuscular injection of a liquid preparation, oral administration of a liquid or solid preparation, or by topical application. Administration can also be accomplished by use of a rectal suppository or a urethral suppository.

The pharmaceutical preparations of the invention can be prepared by known dissolving, mixing, granulating, or tablet-forming processes. For oral administration, the Androgen Receptor Antagonists or their physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the Androgen Receptor Antagonists or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

The preparation of pharmaceutical compositions which contain an active component is well understood in the art. Typically, such compositions are prepared as aerosols of the polypeptide delivered to the nasopharynx or as injectables, either as liquid solutions or suspensions; however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like or any combination thereof.

In addition, the composition can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For topical administration to body surfaces using, for example, creams, gels, drops, and the like, the Androgen Receptor Antagonists or their physiologically tolerated derivatives such as salts, esters, N-oxides, and the like are prepared and applied as solutions, suspensions, or emulsions in a physiologically acceptable diluent with or without a pharmaceutical carrier.

In another embodiment, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

For use in medicine, the salts of the Androgen Receptor Antagonist will be pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic: acid, oxalic acid, citric acid, tartaric acid, carbonic acid or phosphoric acid.

The following examples are presented in order to more fully illustrate the preferred embodiments of the invention. They should in no way be construed, however, as limiting the broad scope of the invention.

### Experimental Details Section

Experimental Methods

Cell Lines

The origins of the cell lines used in the studies described herein are shown in Table 1 below:

TABLE 1

| | | Receptor | | |
|---|---|---|---|---|
| Cell line | Morphology | expressed | Origin | Patient |
| LNCaP | Epithelial | Androgen; | Needle | 50-year-old white male with |
| | | Estrogen | aspiration | stage D1 prostatic cancer |
| | | | biopsy of left | |
| | | | supraclavicular | |
| | | | lymph node | |
| DU 145 | Epithelial | | Metastatic CNS | 69-year-old white male with |
| | | | lesion | metastatic carcinoma of the |
| | | | | prostate and a 3 year history of |
| | | | | lymphocytic leukemia |
| PC-3 | Epithelial | | Prostatic | 62-year-old male Caucasian with |
| | | | metastatic bone | grade IV prostatic |
| | | | marrow | adenocarcinoma |
| PPC-1 | Epithelial | | Transurethral | 67-year-old black male with |
| (primary prostate | | | resection of the | stage D2 poorly differentiated |
| carcinoma-1) | | | prostate | adenocarcinoma of prostate |
| TSU | Epithelial | | Metastatic | 73-year-old male Japanese with a |
| | | | tumor in a | moderately differentiated prostatic |
| | | | cervical lymph | adenocarcinoma |
| | | | node | |
| TCCSUP | Epithelial | | Analpastic | 67-year-old female with grade |
| | | | transitional cell | IV bladder cancer |
| | | | carcinoma in | |
| | | | the neck of the | |
| | | | urinary bladder | |
| HT-29 | Epithelial | Urokinase | Colorectal | 44-year-old female Caucasian |
| | | receptor; | adenocarcinoma | |
| | | Vitamin D | | |
| CV-1 | Fibroblast | | Normal kidney | Male adult (141 days) African |
| | | | | green monkey |
| MCF-7 | | | Breast | |
| | | | adenocarcinoma | |

Cell Growth Conditions

PPC-1, LNCaP, TSU, PC-3, and DU145 were grown in PRMI-1640 medium containing 2 mM L-glutamine supplemented with 10% fetal bovine serum (FBS). Cells were maintained in a 5% CO₂/95% air humidified atmosphere at 37° C.

Sulforhodamine B (SRB) Assay

The SRB Assay was used to determine cell number during cytotoxocity experiments. The following protocol was used:
1. Cells were detached with 0.25% trypsin.
2. Experimental cultures were cultured in 96-well microtiter plates (200 uL growth medium per well; 1,000-200,000 cells per well).
3. Cultures were fixed with 50 uL 50% TCA (4° C.). (see cell fixation protocol for details).
4. Fixed cells were stained with 50 uL 0.4% (wt/vol) SRB in 1% acetic acid for 10 minutes.
5. SRB was removed and the cultures were quickly* rinsed 5 times with 1% acetic acid to remove unbound dye.**
   * quickly performing rinsing process is to prevent desorption of protein-bound SRB
   ** completely remove residual wash solution by sharply flicking plates over sink.
6. Cultures were air-dried overnight until there is no visible moisture.
7. The cellular protein-bound SRB was dissolved with 200 uL unbuffered Tris base (10 mM, pH 10.5) for 30 minutes on a rocking platform shaker.
8. Absorbance was read at 540 nm.
   Fixation of Cells Attached to the Plastic Substratum

The following protocol was used for fixing cells:
a. 50 uL of 50% TCA (4° C.) were gently layered on the top of growth medium in each well to make a final TCA concentration of 10%.
b. Cultures were incubated at 4° C. for 1 hour.
c. Cultures were washed 5 times with tap water to remove TCA, growth medium, low-molecular-weight metabolites, and serum protein.
d. Plates were air-dried until there was no visible moisture.

### Results

### EXAMPLE 1

### Extended Cytotomcity Studies of Compounds in Different Cell Lines

The IC₅₀s of I-31, I-29, S-NTBA and Compound V in different prostate cancer cell lines (DU 145, PC-3, TSU, PPC-l and LNCaP), in a breast cancer cell line (MCF-7), in other cancer cell lines (MCF-7) and in a control cell line (CV-1) after 1 and 4 days of incubation with the drug, are shown in Tables 2A and B.

TABLE 2A
Prostate cancer Cell Lines

| | | drug | | | | | |
|---|---|---|---|---|---|---|---|
| | | incubation | IC₅₀ (uM) | | | | |
| Cpd ID | Structure | time (day) | LNCaP | DU 145 | PC-3 | PPC-1 | TSU |
| I-31 | 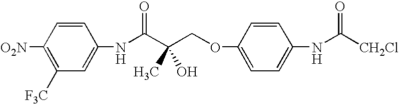 | 1 4 | 2.9 ± 1.2 3.3 ± 0.4 | 1.0 ± 0.2 0.7 ± 0.1 | 5.4 ± 0.8 5.3 ± 0.8 | 1.9 ± 0.1 1.9 ± 0.1 | 1.3 ± 0.1 1.1 ± 0.1 |
| I-29 | 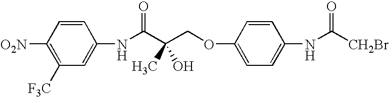 | 1 4 | 1.1 ± 0.4 1.8 ± 0.2 | 0.4 ± 0.1 0.4 ± 0.1 | 2.3 ± 0.1 2.4 ± 0.4 | 0.9 ± 0.1 1.3 ± 0.1 | 1.3 ± 0.1 1.0 ± 0.1 |
| S-NTBA | 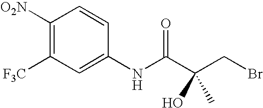 | 1 4 | 1.6 ± 0.5 1.8 ± 0.9 | 5.5 ± 0.6 4.6 ± 0.8 | 7.9 ± 0.7 3.7 ± 1.1 | 1.7 ± 0.2 2.7 ± 0.8 | 4.9 ± 0.4 2.9 ± 0.2 |
| Cmpd V | 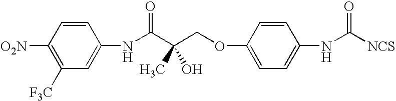 | 1 4 | 19.8 ±10.5 12.8 ±4.1 | 62.1 ±0.6 69.7 ±6.5 | 47.8 ±4.2 48.5 ±10.1 | 63.7 ±4.6 62.6 ± 4.9 | 54.1 ±5.5 55.0 ± 0.8 |

TABLE 2B
Other Cancer Cell Lines and Control

| | | drug | | | |
|---|---|---|---|---|---|
| | | incubation | IC₅₀ (uM) | | |
| Cpd ID | Structure | time (day) | HT-29 | TCCSUP | MCF-7 |
| I-31 | 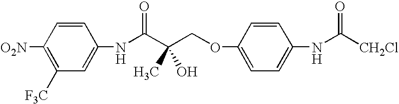 | 1 4 | 5.4 ± 0.1 3.7 ± 0.7 | 2.1 ± 0.3 2.1 ± 0.3 | 15.8 ± 2.7 14.0 ± 1.1 |
| I-29 | 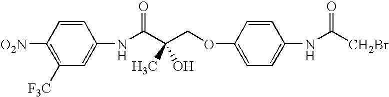 | 1 4 | 3.6 ± 0.4 4.4 ± 0.2 | 2.6 ± 0.4 2.5 ± 0.4 | 11.9 ± 0.3 12.9 ± 0.7 |
| S-NTBA | 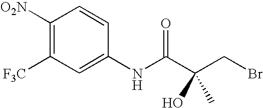 | 1 4 | 19.8 ± 1.0 10.8 ± 0.4 | 9.5 ± 3.6 4.6 ± 0.3 | 3.3 ± 0.6 3.3 ± 0.6 |
| Cmpd V | 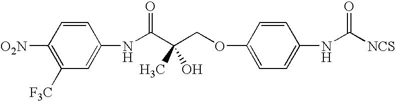 | 1 4 | 85.5 ± 4.5 71.0 ± 1.2 | 90.4 ± 2.2 78.1 ± 11.2 | 32.7 ± 1.9 39.4 ± 5.4 |

### EXAMPLE 2

### Effect of Compounds on Apoptosis in Different Cell Lines

The ability of I-31, I-29, S-NTBA and compound V to induce apoptosis in different prostrate cancer cell lines (DU145, PP-3 TSU, PPC-1 and LNCaP), in a breast cancer cell line(MCF-7) and in other cell lines (TCCSUP, HT-29) and in a control cell line (CV-1) was determined and the results are shown in Table 3.

TABLE 3
Apoptosis by Histone assay

| | | Enrichment factor | | | | |
|---|---|---|---|---|---|---|
| Cpd ID | Structure | LNCaP | DU 145 | PC-3 | PPC-1 | TSU |
| I-31 | 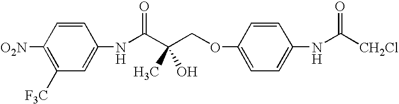 | 1.374 | 2.500 | 0.594 | 1.312 | 0.848 |
| I-29 | 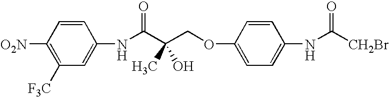 | 16.571 | 18.643 | 0.531 | 1.146 | 1.008 |
| S-NTBA | 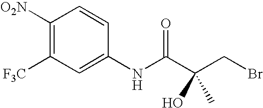 | 0.660 | 1.929 | 0.750 | 0.697 | 0.705 |
| Cmpd V | 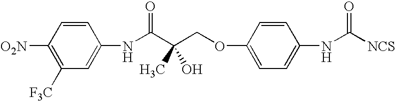 | 2.000 | 5.071 | 2.500 | 1.749 | 1.167 |

| | Enrichment factor | | | |
|---|---|---|---|---|
| Cpd ID | HT-29 | TCCSUP | MCF-7 | CV-1 |

| | | | | |
|---|---|---|---|---|
| I-31 | 1.205 | 0.471 | 0.938 | 0.702 |
| I-29 | 1.667 | 0.000 | 1.617 | 0.786 |
| S-NTBA | 1.538 | 0.118 | 2.012 | 0.393 |
| Cmpd V | 1.410 | 1.176 | 1.926 | 6.548 |

### EXAMPLE 3

### Sensitivity of Androgen Receptor Responsive Cells to Compound V

The relative androgen receptor (AR) expression for various cell lines is shown in Table 4. Both LNCAP prostate carcinoma and MCF-7 breast carcinoma cell lines express androgen receptor, while the prostate carcinoma cell lines PC-3 and Du145 and African green monkey kidney epithelial CV-1 cells do not express androgen receptor (Chlenski, MacIndoe, Warriar, Webber 1 & 2).

TABLE 4
Comparison of Androgen Receptor Expression
for Various Cell Lines

| Cell Line | LNCaP | PC-3 | Du145 | MCF-7 | CV-1 |
|---|---|---|---|---|---|
| AR | + | − | − | + | − |
| Mutated | + | N/A | N/A | − | N/A |
| Mutations | T877A | N/A | N/A | N/A | N/A |
| Methylation of | − | Strong | Full | − | N/A |
| AR Promoter | | | | | |
| Transcription | Normal | − | − | Normal | − |
| PSA expression | + | − | − | + | − |
| Other Steroid | | | | | |
| Receptors: | | | | | |
| EGF/TGF-α | + | + | + | + | ? |
| FGF | + | + | + | + | ? |
| IGF | + | + | + | + | ? |
| TGF-β | − | + | + | + | ? |
| TR | − | + | + | + | + |
| GR | − | + | + | + | Non-functional |
| ER | − | + | + | + | − |
| PR | − | − | + | + | − |

Materials and Methods:

Materials:

DMSO is the vehicle control and the solvent for ICI 182,780 and Compound V. ICI 182,780 is a high affinity estrogen receptor antagonist devoid of any partial agonism both in vitro and in vivo. Compound V is an androgen receptor antagonist.

Methods:

Plated 100,000 (105) cells/well in five 6-well plates, with a total volume of 2.5 mL media/well. The plates were incubated at 37° C., 5% CO2 for 24 h to allow the cells sufficient time to attach and be in log phase growth at the start of the experiment. The media was aspirated from 4 of the plates and replaced with 2.5 mL media containing vehicle control (0.125% DMSO), 0.25 nM ICI 182,780, or 0.25 nM ICI 182,780 in combination with increasing concentrations of Compound V (0.5, 1.0, 5.0, 7.5, 10.0, and 20.0 μM). Three wells were given the same concentration of the drugs or DMSO for each treatment condition listed above. The total volume of DMSO/drug added to each well was equal to 0.125% of the media volume in each well. The cells from the remaining 6-well plate were collected and counted to determine plating efficiency. The 6-well plates containing DMSO/drug were incubated for 120 h at 37° C., 5% CO2. After 120 h, the media from each well was collected along with trypsinized cells and pelleted at 150×g. The cells were resuspended in 600 μl media, from which 90 μl was taken and combined with 10 μl trypan blue for counting on a hemacytometer.

Cell Culture and Reagents:

LNCaP, PC-3, MCF-7, and CV-1 cells were obtained from the American Type Culture Collection (Manassas, Va.) and grown according to ATCC guidelines, with the exception of PC-3 cells. PC-3 cells were adapted to grow in RPMI-1640 medium (Mediatech, Herndon, Va.) supplemented with L-glutamine (Mediatech) and 10% FBS (HyClone, Logan, Utah). LNCaP cells were also adapted to grow in medium containing charcoal-stripped FBS (CS LNCaP). LNCaP cells were grown for 2 weeks in medium with charcoal-stripped FBS to allow sufficient time for adaptation before any experiments were conducted. DMSO (Sigma, St. Louis, Mo.) is the vehicle control and the solvent for Compound V and ICI 182, 780 (Tocris, Ellisville, Mo.). ICI 182,780 is a high affinity estrogen receptor antagonist devoid of any partial agonism both in vitro and in vivo.

Charcoal-Stripped Fetal Bovine Serum:

Steroids were removed from FBS as described previously (Klus et al, 1996). Briefly, 4 mg/mL activated charcoal was added to the serum and stirred for 16 h at 4° C. The serum was centrifuged for 1 h at 4oC at 1600×g. The supernatant was collected and centrifuged for 1.5 h at 4° C. at 25,000 rpm in Optima LE-80K ultracentrifuge. The supernatant was then filtered through a 0.22 μM filter and stored in 100 mL aliquots at −20° C.

Cell Viability Assay:

Cells were plated at 100,000 (105) cells/well in five 6-well plates, with a total volume of 2.5 mL media/well. The plates were incubated at 37° C., 5% CO2 for 24 h to allow the cells sufficient time to attach and be in log phase growth at the start of the experiment. The media was aspirated from 4 of the plates and replaced with 2.5 mL media containing vehicle control (DMSO) or drug dissolved in DMSO. The total volume of DMSO/drug added to each well was equal to 0.1% of the media volume in each well. LNCaP, CS LNCaP, PC-3, MCF-7, and CV-1 cells were treated with vehicle control, and increasing concentrations of Compound V (0.01, 0.05, 0.1, 0.5, 1.0, 5.0, and 10.0 μM). In addition, MCF-7 cells were treated with vehicle control, 0.25 nM ICI 182,780, or 0.25 nM ICI 182,780 in combination with increasing concentrations of Compound V (0.5, 1.0, 5.0, 7.5, 10.0, and 20.0 μM). Three wells were given the same concentration of the drugs or DMSO for each treatment condition listed above. The cells from the remaining 6-well plate were collected and counted to determine plating efficiency. The 6-well plates containing DMSO/drug were incubated for 120 h at 37° C., 5% CO2. After 120 h, the media from each well was collected along with trypsinized cells and centrifuged at 150×g. The cells were resuspended in 600 μl of media, from which 90 μl was taken and combined with 10 μl trypan blue (Mediatech) for counting on a hemacytometer.

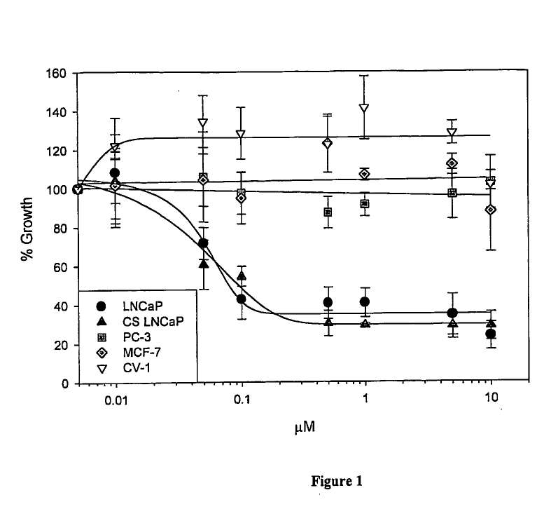

Several of these cell lines were tested for sensitivity to Compound V, as well as LNCaP cells that were cultured for 2 weeks in media containing charcoal-stripped FBS (steroid hormone free) (CS LNCaP) (FIG. 1). Cells were plated at 100,000 (105) cells/well in five 6-well plates, with a total volume of 2.5 mL media/well. The plates were incubated at 37° C., 5% CO2 for 24 h to allow the cells sufficient time to attach and be in log phase growth at the start of the experiment. The media was aspirated from 4 of the plates and replaced with 2.5 mL media containing vehicle control (DMSO) or drug dissolved in DMSO. The total volume of DMSO/drug added to each well was equal to 0.1% of the media volume in each well. LNCaP, CS LNCaP, PC-3, MCF-7, and CV-1 cells were treated with vehicle control, and increasing concentrations of Compound V (0.01, 0.05, 0.1, 0.5, 1.0, 5.0, and 10.0 μM). Three wells were given the same concentration of the drugs or DMSO for each treatment condition listed above. The cells from the remaining 6-well plate were collected and counted to determine plating efficiency. The 6-well plates containing DMSO/drug were incubated for 120 h at 37° C., 5% CO₂. After 120 h, the media from each well was collected along with trypsinized cells and centrifuged at 150×g. The cells were resuspended in 600 μl of media, from which 90 μl was taken and combined with 10 μl trypan blue (Mediatech) for counting on a hemacytometer.

Results:

Cell lines that do not express the AR (PC-3 and CV-1) are not sensitive to Compound V, indicating that AR expression is required for growth inhibition (FIG. 1). Although MCF-7 cells express the AR, the AR pathway is a secondary growth pathway and not required for growth, so MCF-7 cells are not sensitive to Compound V. Both LNCaP and CS LNCAP are sensitive with IC₅₀ values of 0.08 and 0.09 μM, respectively, and maximal growth inhibition at 65% and 70% as compared to vehicle control treated cells.

In order to determine the importance of blocking the androgen receptor pathway in MCF-7 cells, the estrogen receptor pathway had to be inhibited in order to block the primary growth signaling pathway. ICI 182, 780 is a highly selective estrogen receptor antagonist devoid of any partial agonism both in vitro and in vivo. MCF-7 cells were treated for 120 h with increasing concentrations of ICI 182, 780 in order to find a dose that was <20% growth inhibitory as compared to MCF-7 cells treated with vehicle control (data not shown). MCF-7 cells were then treated with vehicle control, 0.25 nM ICI 182,780, or 0.25 nM ICI 182,780 in combination with increasing concentrations of Compound V (0.5, 1.0, 5.0, 7.5, 10.0, and 20.0 μM).

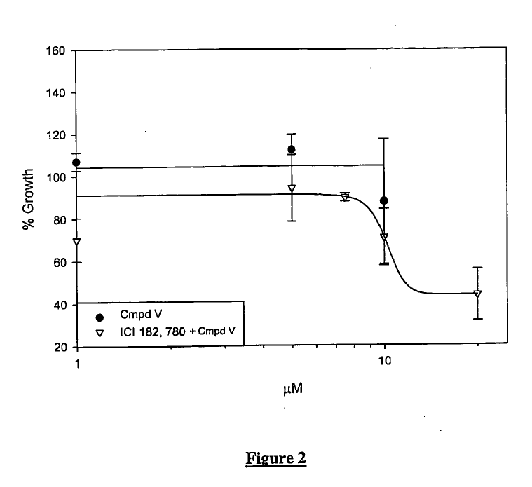

As shown in FIG. 2, once the estrogen receptor pathway was blocked, MCF-7 cells became sensitive to Compound V, although at a higher IC₅₀ of 11.5 μM and maximal growth inhibition at 56%. This demonstrates that Compound V is working through the androgen receptor pathway, and that this is a secondary growth pathway for the MCF-7 breast carcinoma cells.

It will be appreciated by a person skilled in the art that the present invention is not limited by what has been particularly shown and described hereinabove.

1. A method of treating a subject suffering from breast cancer, comprising the step of administering to said subject an Androgen Receptor Antagonist, in an amount effective to treat breast cancer in said subject.

2. The method according to claim 1, comprising administering an an analog, derivative, isomer, metabolite, pharmaceutically acceptable salt, pharmaceutical product, hydrate, N-oxide, crystal, polymorph or prodrug of said Androgen Receptor Antagonist, or any combination thereof

3. The method of claim 1, wherein said Androgen Receptor Antagonist is an alkylating agent.

4. The method of claim 1, wherein said Androgen Receptor Antagonist is an alkylating agent which binds irreversibly to an androgen receptor.

5. The method according to claim 1, wherein said Androgen Receptor Antagonist is represented by the structure of formula I: 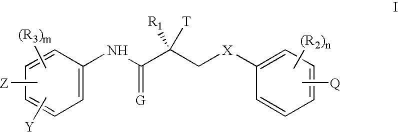
X is a bond, O, CH₂, NH, S, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 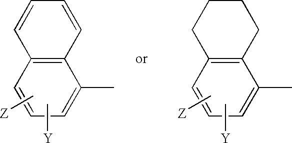
Z is NO₂, CN, COR, COOH, or CONHR;
Y is CF₃, F, Br, Cl, I, CN, or SnR₃;
Q is SCN, NCS, OCN, or NCO;
n is an integer of 1-4; and
m is an integer of 1-3.

6. The method of claim 5, wherein G is O, T is OH, R1 is CH3, X is O, Z is NO2, Y is CF3, and Q is NCS.

7. The method according to claim 1, wherein said Androgen Receptor Antagonist is represented by the structure of formula II. 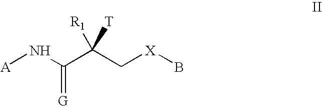
wherein X is a bond, O, CH₂, NH, S, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from: 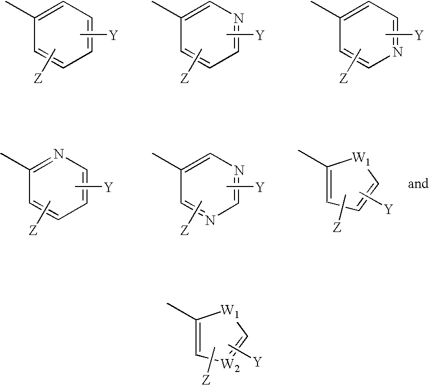
B is a ring selected from: 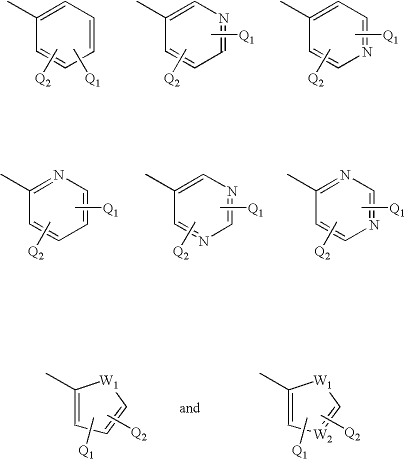
wherein A and B cannot simultaneously be a benzene ring;
Z is NO₂, CN, COOH, COR, NHCOR or CONHR;
Y is CF₃, F, I, Br, Cl, CN CR₃ or SnR₃;
Q₁ is NCS, SCN, NCO or OCN;
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 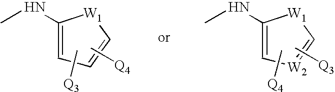
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO.

8. The method according to claim 7, wherein G is O, T is OH, R1 is CH3, X is O, Z is NO2, Y is CF3, and Q1 is NCS.

9. The method according to claim 1, wherein said Androgen Receptor Antagonist is represented by the structure of formula III. 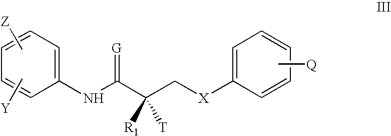
wherein X is a bond, O, CH₂, NH, S, Se, PR, NO or NR;
G is O or S;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is SCN, NCS, OCN, or NCO;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

10. The method according to claim 9, wherein G is O, T is OH, R1 is CH3, X is O, Z is NO2, Y is CF3, and Q is NCS.

11. The method according to claim 1, wherein said Androgen Receptor Antagonist is represented by the structure of formula IV: 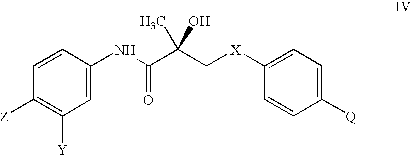
wherein X is a bond, O, CH₂, NH, S, Se, PR, NO or NR;
Z is NO₂, CN, COOH, COR, NHCOR or CONHR;
Y is CF₃, F, I, Br, Cl, CN, CR₃ or SnR₃;
Q is SCN, NCS, OCN, or NCO; and
R is alkyl, haloalkyl, dihaloalkyl, trihaloalkyl, CH₂F, CHF₂, CF₃, CF₂CF₃, aryl, phenyl, halogen, alkenyl or OH.

12. The method according to claim 1, wherein said Androgen Receptor Antagonist is represented by the structure of formula V:

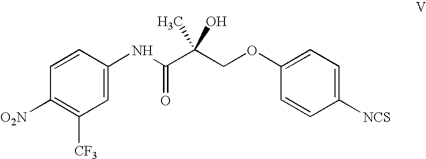

13. The method according to claim 1, wherein said subject is a female subject.

14. The method according to claim 1, wherein said subject is a male subject.

15. The method according to claim 1, wherein said administering comprises administering a pharmaceutical preparation comprising said Androgen Receptor Antagonist and/or its analog, derivative, isomer, metabolite, pharmaceutically acceptable salt, pharmaceutical product, hydrate, N-oxide, crystal, polymorph, prodrug or any combination thereof; and a pharmaceutically acceptable carrier.

16. The method according to claim 15, wherein said administering comprises intravenously, intra-arterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

17. The method according to claim 15, wherein said pharmaceutical preparation is a pellet, a tablet, a capsule, a solution, a suspension, an emulsion, an elixir, a gel, a cream, a suppository or a parenteral formulation.

18. A method of preventing, suppressing, inhibiting or reducing the incidence of breast cancer in a subject, comprising the step of administering to said subject an Androgen Receptor Antagonist, in an amount effective to prevent, suppress, inhibit or reduce the incidence of breast cancer in said subject.

19. The method according to claim 18, comprising administering an an analog, derivative, isomer, metabolite, pharmaceutically acceptable salt, pharmaceutical product, hydrate, N-oxide, crystal, polymorph or prodrug of said Androgen Receptor Antagonist, or any combination thereof.

20. The method of claim 18, wherein said Androgen Receptor Antagonist is an alkylating agent.

21. The method of claim 18, wherein said Androgen Receptor Antagonist is an alkylating agent which binds irreversibly to an androgen receptor

22-102. (canceled)
